(19) United States
(12) Patent Application Publication
(10) Pub. No.: US 2010/0094412 A1
(43) Pub. Date: Apr. 15, 2010

(54) Accommodating intraocular lens

(76) Inventor: Doug Wensrich, Bedford, TX (US)

Correspondence Address: ALCON, IP LEGAL, TB4-8, 6201 SOUTH FREEWAY, FORT WORTH, TX, 76134, US

(21) Appl. No.: 12/578,713

(22) Filed: Oct. 14, 2009

Related U.S. Application Data
(60) Provisional application No. 61/105,517, filed on Oct. 15, 2008.

(51) Int. Cl. A61F 2/16

(52) U.S. Cl. 623/6.13; 623/6.32; 623/6.38

(57) ABSTRACT

An improved multifocal design for an ocular implant is provided. This ocular implant can include an accommodating intraocular lens (IOL) and a number of haptics. The accommodating IOL includes a liquid suspended between two optically transparent plates or membranes to form a pressure lens that passes optical energy. The haptics mechanically couple to the IOL in order to position and secure the IOL within the eye. The IOL achieves accommodation by using the eye's ciliary muscles to vary the surface curvature of the liquid. The liquid may have a high surface tension and be surrounded by phobic liquid. Pressure from the ciliary muscles causes fluid to be added from or withdrawn to a reservoir. Increasing/decreasing the internal pressure of the liquid changes the angle (curvature) of the surface thus changing the optical properties of the lens. When the pressure is released the liquid returns to the reservoir. The whole system may be sealed off from the interior of the eye by a membrane/transparent lens.

This application claims priority to U.S. Provisional Application Ser. No. 61/105,517 filed on Oct. 15, 2008.

### TECHNICAL FIELD OF THE INVENTION

The present invention relates generally to the human eye and more particularly to intraocular lenses (IOLs).

### BACKGROUND OF THE INVENTION

The human eye in its simplest terms functions to provide vision by transmitting light through a clear outer portion called the cornea, and focusing the image by way of a lens onto a retina. The quality of the focused image depends on many factors including the size and shape of the eye, and the transparency of the cornea and lens. Age and/or disease often cause the lens to become less transparent. Thus, vision deteriorates because of the diminished light which can be transmitted to the retina. This deficiency in the lens of the eye is medically known as a cataract.

An accepted treatment for this condition is surgical removal of the lens and replacement of the lens function by Intraocular lenses (IOLs). IOLs are the artificial lenses that replace the eye's natural lens that is removed during cataract surgery. For many years most IOLs were made of poly (methylmethacrylate), a material with good optical characteristics and compatibility with the tissues of the eye. A disadvantage of PMMA is, however, that it is a very rigid material and the incision must be made large enough for implantation of the IOL. If the optical properties are not correctly matched, a need for a second IOL is required.

Traditional IOLs are monofocal, meaning these lenses offer vision at one distance only (far, intermediate or near). Traditional IOLs offer an improvement over the cataractous lens that is replaced during surgery, which provides only cloudy, blurred vision at any distance. But traditional IOLs mean that the patient must wear eyeglasses or contact lenses in order to read, use a computer or view objects at the unselected distance. There is still a need for multifocal and accommodating IOLs that offer the patient the possibility of seeing well at more than one distance, without glasses or contacts.

### SUMMARY OF THE INVENTION

Embodiments of the present invention provide an improved ocular implant. This ocular implant includes an accommodating intraocular lens (IOL) and a number of haptics. The accommodating IOL includes a liquid suspended between two optically transparent plates or membranes to form a pressure lens that passes optical energy. The haptics mechanically couple to the IOL in order to position and secure the IOL within the eye. The IOL achieves accommodation by using the eye's ciliary muscles to vary the surface curvature of the liquid. The liquid may have a high surface tension and be surrounded by phobic liquid. Pressure from the ciliary muscles causes fluid to be added from or withdrawn to a reservoir. Increasing/decreasing the internal pressure of the liquid changes the angle (curvature) of the surface, thus changing the optical properties of the lens. When the pressure is released the liquid returns to the reservoir. The whole system may be sealed off from the interior of the eye by a membrane/transparent lens.

The ocular implant is operable to be implanted within a reduced sized incision of the capsular bag of an eye. This IOL includes a foldable optic and a number of haptics coupled to the optic. In one embodiment the haptics are multi hinged while another embodiment allows the haptics to be placed at an angle to the plane of the optic. The haptics flex while minimizing buckling and vaulting of the IOL in order to position and secure the IOL within the eye.

The IOL provided is made from a foldable optic. This allows the IOL to be implanted within a reduced sized incision. The haptics coupled to the IOL position the IOL within the capsular bag of an eye. The haptics may be multi hinged, oriented at an angle relative to the optic, or combination of the two. The optic may have an edge of less than about 0.15 millimeters.

Another embodiment of the present invention provides a method to correct the visual impairment of an aphakia. This method involves removal of a natural lens from an eye. An IOL is inserted during an incision in the capsular bag of the eye. As discussed previously, the IOL can accommodate both near and far vision. This is accomplished using a pressure lens that interfaces the IOL to the ciliary muscles of the eye. The haptics mechanically couple to the IOL in order to position and secure the IOL within the eye.

Other advantages of the present invention will become more apparent to one skilled in the art upon reading and understanding the detailed description of the preferred embodiments described herein with reference to the following drawings.

### BRIEF DESCRIPTION OF THE DRAWINGS

For a more complete understanding of the present invention and the advantages thereof, reference is now made to the following description taken in conjunction with the accompanying drawings in which like reference numerals indicate like features and wherein:

FIG. 1 illustrates the anatomy of the eye in which an IOL in accordance with embodiments of the present invention may be implanted;

FIG. 2 depicts an IOL in accordance with embodiments of the present invention;

FIGS. 3A and 3B provides a top down view and a cross section of IOL in accordance with embodiments of the present invention;

FIGS. 4A and 4B provide a top down view and a cross section of IOL in accordance with embodiments of the present invention;

FIGS. 5A and 5B provide a top down view and a cross section of IOL in accordance with embodiments of the present invention;

FIGS. 6A and 6B provide a cross section of IOL that depicts how the IOL may adjust using pressure from the ciliary muscles in accordance with embodiments of the present invention;

FIG. 7 provides a cross section of IOL that depicts how the IOL may adjust using pressure to adjust fluid pressure within the IOL to shape the IOL in accordance with embodiments of the present invention;

FIG. 8 provides a logic flow diagram of a method to correct for visual impairments such as aphakia of the eye in accordance with embodiments of the present invention; and

FIG. 9 provides a logic flow diagram of a method in which an IOL uses a pressure lens to accommodate for near and far distance in accordance with embodiments of the present invention.

### DETAILED DESCRIPTION OF THE INVENTION

Preferred embodiments of the present invention are illustrated in the FIGs., like numerals being used to refer to like and corresponding parts of the various drawings.

An improved design for an ocular implant is provided by embodiments of the present invention. This ocular implant includes an accommodating intraocular lens (IOL) and a number of haptics. The accommodating IOL includes a liquid suspended between two optically transparent plates or membranes where the IOL passes optical energy. The haptics mechanically couple to the IOL in order to position and secure the IOL within the eye. The IOL achieves accommodation by using the eye's ciliary muscles to vary the surface curvature of an amount (e.g., a drop) of the liquid. The liquid has a high surface tension and is surrounded by phobic liquid. Pressure from the ciliary muscles causes fluid from a reservoir to be added to or withdrawn from the drop. Increasing/decreasing the size of the drop changes the angle of the surface, thus changing the index of refraction. When the pressure is released, the liquid returns into the reservoir. The surrounding liquid is used to increase the stability of the suspended drop. The surrounding liquid can flow into and out of a second reservoir as the liquid droplet increased or decreased in size. The whole system can be sealed off from the interior of the eye by a membrane/transparent lens.

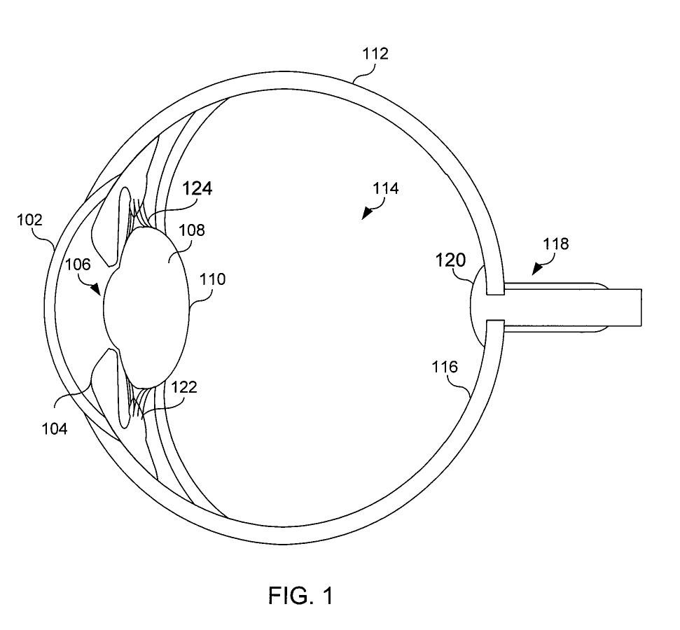

Sight is, by far, one of our most valuable senses. Without our vision, everyday tasks like driving and reading books would be impossible. Our eyes are complex machines that deliver a clear picture of the world around us—communicating the simplest of colors, shapes and textures. FIG. 1 illustrates the anatomy of the eye into which the improved design for ocular implant provided by the present invention may be placed. Eye 100 includes cornea 102, iris 104, pupil 106, lens 108, lens capsule 110, zonules, ciliary body 120, sclera 112, vitreous gel 114, retina 116, macula, and optic nerve 120. Cornea 102 is a clear, dome-shaped structure on the surface of the eye acts as a window, letting light into the eye. Iris 104 is the colored part of the eye, called the iris, is a muscle surrounding the pupil that relaxes and contracts to control the amount of light entering the eye. Pupil 106 is the round, central opening of the iris. Lens 108 is the structure inside the eye that, in combination with the cornea, operates to focus light on the retina. Lens capsule 110 is an elastic bag that envelops the lens, helping to control lens shape when the eye focuses on objects at different distances. Zonules are slender ligaments that attach the lens capsule to the inside of the eye, holding the lens in place. The Ciliary body is the muscular area attached to the lens that contracts and relaxes to control the size of the lens for focusing. Sclera 112 is the tough, outermost layer of the eye that maintains the shape of the eye. Vitreous gel 114 is the large, gel-filled section that is located towards the back of the eyeball, and which helps to maintain the curvature of the eye. Retina 116 is a light-sensitive nerve layer in the back of the eye that receives light and converts it into signals to send to the brain. The macula is the area in the back of the eye that contains functions for seeing fine detail. Optic nerve 118 connects and transmits signals from the eye to the brain.

Ciliary body 122 lies just behind the iris 104. Attached to the ciliary body 122 are tiny fiber “guide wires” called zonules 124. Lens 108 is suspended inside the eye by the zonular fibers 124. Nourishment for the ciliary body 122 comes from blood vessels which also supply the iris 104. One function of ciliary body 122 is to control accommodation by changing the shape of the lens 108. When the ciliary body 122 contracts, the zonules 124 relax. This allows the lens 108 to thicken, increasing the eye's ability to focus up close. When looking at a distant object, ciliary body 122 relaxes, causing the zonules 124 to contract. The lens 108 then becomes thinner, adjusting the eye's focus for distance vision. Embodiments of the present invention provide an IOL that uses these functions of the ciliary body to control accommodation of the IOL by changing the shape of the IOL by changing the internal pressure of a fluid within the IOL lens.

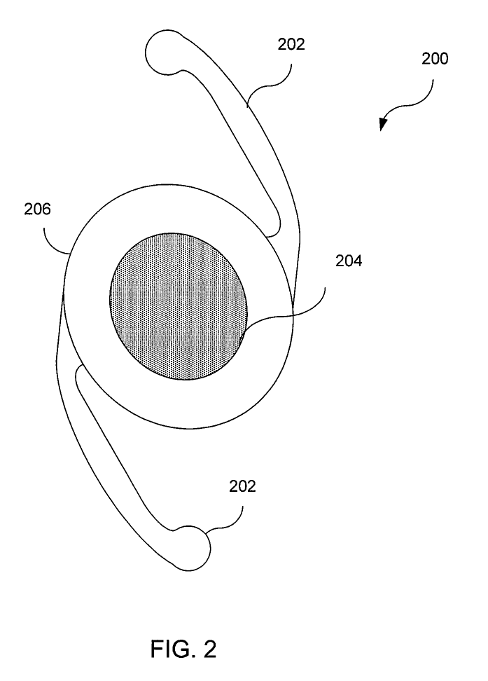

FIG. 2 depicts an IOL 200. IOL 200 is an artificial lens implanted in the eye to restore vision after a natural lens has been removed. The need for the IOL may be due to cataract, disease or accidents. The lens of the IOL may be convex on both sides (biconvex) and made of a soft plastic, such as the Acrysof material manufactured by Alcon Laboratories, Inc., of Fort Worth, Tex., that can be folded prior to insertion, allowing placement through an incision smaller than the optic diameter of the lens. After surgical insertion into the eye, the lens gently unfolds to restore vision. The supporting arms (haptics) 202 provide for proper positioning of the IOL within the eye.

IOL 200 may be positioned in the posterior chamber of the eye, replacing the natural lens. This position allows IOL 200 to correct the visual impairment of aphakia (absence of the natural lens). IOL 200 may have a biconvex optic that is shaped to provide increased depth of focus. IOL 200 can provide good near, intermediate and distance vision with increased independence from glasses in patients who have undergone cataract surgery. IOL 200 can deliver quality vision for various lighting situations. The central portion 204 may be a pressure lens whose shape may be changed by using the ciliary muscles to adjust the accommodation of the lens. Thus, IOL 200 can accommodate both near and distant focal points.

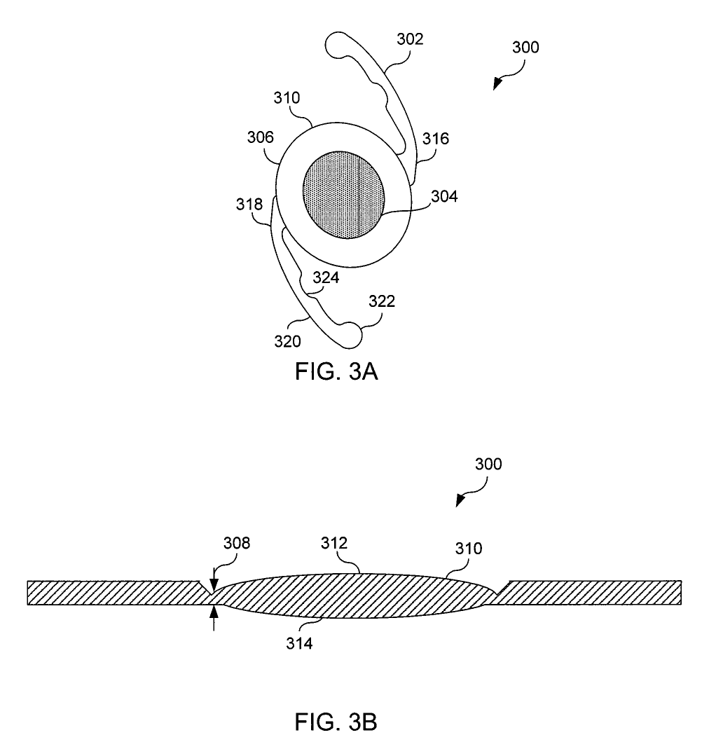

FIGS. 3A and 3B provide a top down view and a cross section of IOL 300 in accordance with embodiments of the present invention. IOL 300 provided is an artificial lens implanted in the eye to restore vision after a natural lens has been removed. IOL 300 is operable to be folded and delivered into the capsular bag through a sub 2.1 mm incision and is optically stable after implantation. The need for the IOL may be due to cataract, disease or accidents. In the relaxed position the lens of the IOL 300 may be convex on both sides (biconvex) and made of a soft plastic that can be folded prior to insertion, allowing placement through an incision smaller than the optic diameter of the lens. After surgical insertion into the eye, the lens gently unfolds to restore vision. The supporting arms (haptics) 302 provide for proper positioning of the IOL within the eye.

Initial alterations to prior IOLs that would allow for implantation through a reduced incision resulted IOLs that were non-optically stable after implantation. These prior attempts merely decreased the thickness of the optic and haptics. This created an unstable optic. Embodiments of the present invention provide unique features that result in an optically stable IOL in the compressed state. These features may be implemented in various combinations and include: (1) a reduced nominal optic edge 308 less than about 0.15 mm; (2) angulated haptic/optic planes; (3) ensuring that any vaulting of optic 306 will occur posteriorly (it might be expected that the lens would vault in the anterior direction because of the angle of the haptics compared to the optic; however, the design actually creates an unexpected, non-vaulting lens; and (4) a multi (double) hinged haptic design. These features result in an optically sound and stable IOL when compressed to 10 mm or 9 mm, while maintaining acceptable force (3.0E-04 N) in the haptics.

IOL 300 may be positioned in the posterior chamber of the eye, replacing the natural lens. This position allows IOL 300 to correct the visual impairment of aphakia (absence of the natural lens). IOL 300 may have a biconvex optic. IOL 300 delivers quality vision for various lighting situations.

In brightly lit conditions, the central portion 304 sends light waves simultaneously to both near and distant focal points, while, in dimly lit conditions, the surrounding area 306 sends greater energy to distance vision.

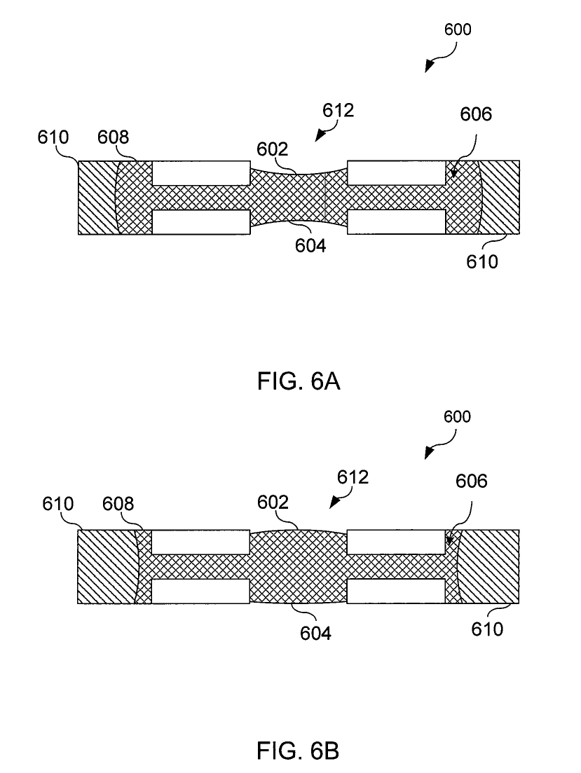

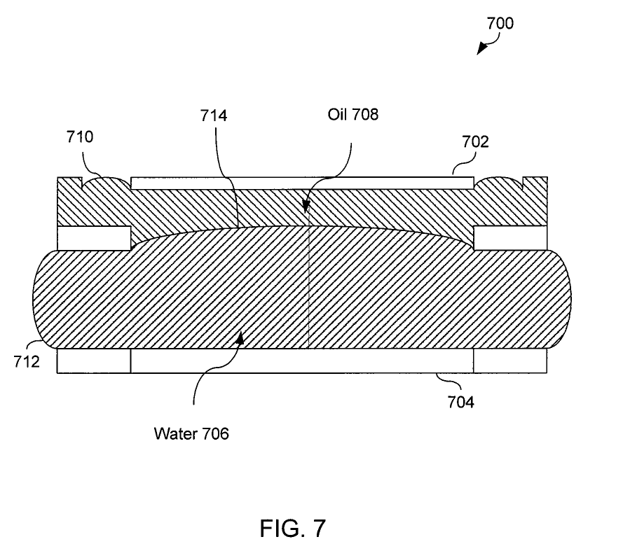

Haptics 302 may be molded in a single piece from the same material as optics 304 and 306. The material used to make IOL 300 may be any soft biocompatible material capable of being folded. Suitable materials are the hydrogel, silicone or acrylic materials described in U.S. Pat. Nos. 5,411,553 (Gerace, et al.), 5,403,901 (Namdaran, et al.), 5,359,021 (Weinschenk, III, et al.), 5,236,970 (Christ, et al.), 5,141,507 (Parekh) and 4,834,750 (Gupta). Optic 310 has an anterior side 314 and a posterior side 312 and may be of any suitable diameter, with between 4.5 mm and 7.0 mm being preferred and 5.5 mm being most preferred. Optic 310 may also be elliptical or oval. The initial thickness of optic 310 will vary depending on the dioptic power desired and the index of refraction for the material used, but generally will be between 0.4 mm and 1.5 mm. Further, the range of optic thickness will vary depending on the ability of the ciliary muscles to exert and relax pressure within the optic as will be discussed with reference to FIGS. 6A, 6B and 7.

IOL 300 provides for a greater diameter of optic 310 while minimizing the size of the surgical incision. The material used to make optic 310 may be modified to absorb ultraviolet radiation, or any other desired radiation wavelength.

Embodiments of haptics 302 can contain gusset 316, first elbow 318, second elbow 324 and distal portion 320 having widened portion 322. In one embodiment, the thickness of first elbow 318, second elbow 324 and distal portion 320 of haptic 302 is uniform, and preferably between about 0.30 mm and 0.60 mm, with between about 0.40 mm and 0.50 mm being more preferred and about 0.43 being most preferred. Gusset 316, however, has a thickness that is reduced toward anterior side 312 of optic 310. Gusset 316 preferably is between about 0.15 mm and 0.60 mm thick, with between about 0.25 mm and 0.35 mm thick being more preferred and about 0.30 mm being most preferred. This reduced thickness generally extends from edge 308 of optic 310. The relatively thin cross section of gusset 316 and edge 308 provides a thinner profile when IOL 300 is inserted through the surgical incision. The reduced thickness of gusset 316 also facilitates fluid circulation (e.g., viscoelastic) between posterior side 314 and anterior side 312 of IOL 300. Alternatively, gusset 316 or optic 310 may be provided with other means (such as holes, grooves, notches, micro-fenestration, or protuberances (all not shown)) to facilitate fluid flow between posterior side 314 and anterior side 312 of IOL 300. The relatively long length and radius of distal portion 320 provides greater contact with the capsular bag for better fixation when IOL 300 is implanted in the eye. First elbow 318 and second elbow 324 create hinges that allow haptic 302 to flex while minimizing buckling and vaulting of optic 310. Widened portion 322 increases the stiffness of haptic 302 just past elbow 318, thereby increasing the strength of haptic 302 at a critical stress point.

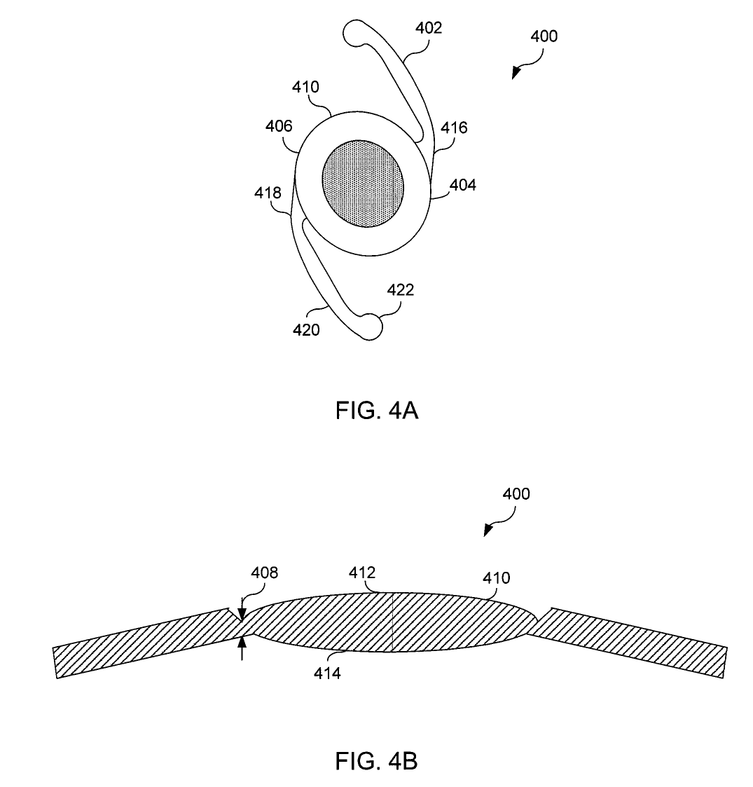

FIGS. 4A and 4B provide a top down view and a cross section of IOL 400 in accordance with embodiments of the present invention similar to that provided in FIGS. 3A and 3B. Haptics 402 contain gusset 416, elbow 418 and distal portion 420 having widened portion 422. In this embodiment the haptics are angulated relative to the plane of the optic. The angulated haptic/optic planes are not parallel. One embodiment angles these planes at about 2.2°. The orientation of these planes ensures that any vaulting of optic 410 will occur posteriorly. Certain embodiments result in a non-vaulting lens (when compressed to about, e.g., 10 mm).

The relatively long length and radius of distal portion 420 provides greater contact with the capsular bag for better fixation when IOL 400 is implanted in the eye. Elbow 418 creates a hinge allowing haptic 402 to flex while minimizing buckling and vaulting of optic 410. Widened portion 422 increases the stiffness of haptic 402 just past elbow 418, thereby increasing the strength of haptic 402 at a critical stress point.

Advantages of embodiments of the present invention provide for: (1) an IOL that can be folded and delivered into the capsular bag through a sub 2.1 mm incision; (2) a single-piece design that represents a significant reduction in IOL volume without sacrificing mechanical stability; and (3) an IOL that can be fabricated as one piece.

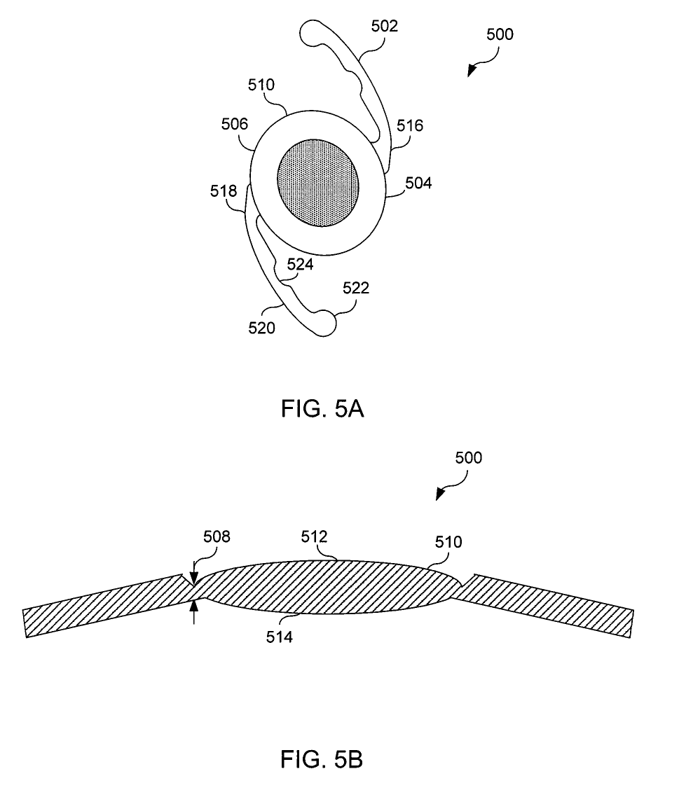

FIGS. 5A and 5B provides a top down view and a cross section of an IOL 500 in accordance with embodiments of the present invention incorporating elements provided in FIGS. 3A, 3B, 4A and 4B. Haptics 502 contain gusset 516, first elbow 518, second elbow 524 and distal portion 520 having widened portion 522. In this embodiment the haptics are multi-hinged and angulated relative to the plane of the optic. The angulated haptic/optic planes are not parallel. One embodiment angles these planes at about 2.2°. The orientation of these planes ensures that any vaulting of optic 510 will occur posteriorly. Certain embodiments result in a non-vaulting lens (when compressed to about, e.g., 10 mm).

The relatively long length and radius of distal portion 520 provides greater contact with the capsular bag for better fixation when IOL 500 is implanted in the eye. First elbow 518 and second elbow 524 create hinges allowing haptic 502 to flex while minimizing buckling and vaulting of optic 510. Widened portion 522 increases the stiffness of haptic 502 just past first elbow 518, second elbow 524, thereby increasing the strength of haptic 502 at a critical stress point.

FIGS. 6A and 6B provide a cross section of an IOL 600 that depicts how the IOL 600 may be adjusted using pressure from the ciliary muscles to alter the shape of the lens 612 in accordance with embodiments of the present invention. Lens 612 includes an upper plate or membrane 602 and lower plate or membrane 604. These membranes should be optically transparent. Additionally, if a liquid with a high enough surface tension is used, the membranes can be the surface of the liquid. The lens 612 is filled with a liquid such as water or oil having appropriate optical characteristics that allow it to pass light with a desired refractive index. Reservoir 608 is shown at either end of the lens 612 and allows ciliary muscles 610 to control or accommodate the shape of the lens 612 by pushing and/or pulling on a diaphragm bounding the reservoir 608. For example, as shown in FIG. 6A, as the ciliary muscles 610 pull at reservoir 608, fluid 606 may be pulled from the lens 612 causing the surfaces or membranes 602 and 604 to have a concave shape. FIG. 6B depicts the same wherein the ciliary muscles 610 push on the reservoir diaphragm causing the fluid pressure within the lens 612 to increase making the lens 612 convex instead of concave. Other embodiments, instead of having an actual plate or membrane, may employ a high surface tension fluid, thus eliminating the need for such a membrane. The pressure lens provided by embodiments of the present invention allow a fluid suspended between plates or within a hole to be flexed as a diaphragm pushes or pulls fluid to or from the liquid between the plates or in the hole.

FIG. 7 provides a cross section of an IOL that depicts how fluid pressure can be used to shape the IOL in accordance with embodiments of the present invention. Accommodating pressure lens 700 includes an upper plate 702, a base plate 704, a first liquid 706, a second liquid 708, a first diaphragm 712 associated with the first liquid 706 and a second diaphragm 710 associated with the second liquid 708. As discussed previously, ciliary muscles may exert pressure by either pushing or pulling on the diaphragms 710 and 712. As the diaphragms are pushed or pulled the internal pressure of liquids 706 and 708 changes, causing the interface 714 between the two liquids to change. This causes a curvature of lens 700 at interface 714 between liquids 706 and 708 to change. In this manner, the ciliary muscles may be used to adjust (accommodate) the pressure lens provided at the interface 714 between the two liquids to allow for both near and far vision.

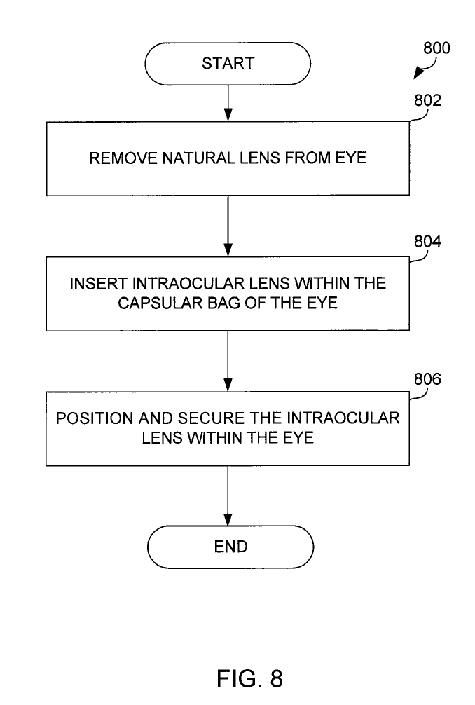

FIG. 8 provides a logic flow diagram of a method to correct for visual impairments such as aphakia of the eye. Operations 800 begin with the removal of a natural lens from an eye in Step 802. The IOL, which may be a multi-focal or accommodating IOL, may then be inserted within the eye. The lenses of the IOL may be convex on both sides (bi-convex) and made of a soft plastic that can be folded prior to insertion. This folding allows placement through a reduced-size incision wherein the incision is smaller than the optic diameter of the IOL. After surgical insertion into the eye in step 804, the IOL may gently unfold to restore vision. In Step 806, the IOL is positioned and secured within the eye. This may be done with the use of supporting arms (haptics) to provide for proper positioning of the IOL within the eye. Embodiments of the present invention may place or position the IOL in posterior chamber of the eye to replace the natural lens as shown in FIG. 1. This position allows the IOL to correct visual impairments such as the absence of a natural lens. The lens itself may be a multi-focal IOL as discussed previously. This can provide patients good near, intermediate and distance vision, and thus provide independence from spectacles following surgery to remove their natural lens.

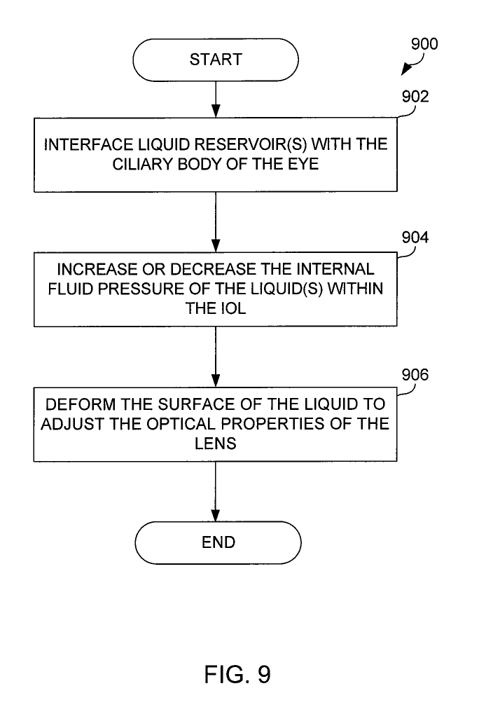

FIG. 9 provides a logic flow diagram of a method in which an IOL uses a pressure lens to accommodate to provide near and far vision in accordance with embodiments of the present invention. Operations 900 begin after the IOL has been implanted in the eye, wherein the IOL may have been placed within the capsular bag of the eye. The IOL has liquid reservoirs that interface with the ciliary body of the eye in Step 902. The interface made in Step 902 allows the ciliary bodies to increase or decrease the internal fluid pressure of the liquids within the IOL based on the relaxation or contraction of the ciliary muscles in Step 904. This change in pressure results in the deformation of the surface of the liquid in Step 906. This deformation adjusts the curvature or optical properties of the lens. By increasing or decreasing the internal fluid pressure, the IOL may accommodate to provide both near and far distance vision.

Accommodating IOLs can be generally grouped into three classes: (1) dynamic single optic (limited range and image quality); (2) dynamic multioptic (sizing and long term reliability issues); and (3) shape changing optic (capsule coupling, reliable distance focus issues). The interaction of shape-changing IOLs with the lens capsule and ciliary body is of special interest. Embodiments may couple the capsule to the IOL to allow single-optic, multiple-optic and shape changing IOLs. This may be done using inherent protein adhesion and augmented with biointegration and supplementary adhesives. The IOL may be constructed using materials with high elasticity like that of the capsule. The IOL may also include a biomimetic scaffold that stimulates tissue integration for shape changing IOL applications. The biomimetic scaffold takes advantage of inherent protein adhesion mechanisms, cellular cues at the capsular interface. Substrate material and surface topography/morphology, chemistry, and biological factors can be tailored to interact with the capsular bag environment so as to stimulate long term cellular integration of the biomimetic scaffold with the lens capsule.

In summary, embodiments of the present invention provide an improved lens design for an ocular implant. This ocular implant includes an accommodating intraocular lens (IOL) and a number of haptics. Embodiments of the accommodating IOL can include a liquid suspended between two optically transparent plates or membranes to form a pressure lens that passes optical energy. The haptics mechanically couple to the IOL in order to position and secure the IOL within the eye. The IOL achieves accommodation by using the eye's ciliary muscles to vary the surface curvature of the liquid. The liquid may have a high surface tension and be surrounded by phobic liquid. Pressure from the ciliary muscles causes fluid to be added from or withdrawn to a reservoir. Increasing/decreasing the internal pressure of the liquid changes the angle (curvature) of the liquid (lens) surface, thus changing the optical properties of the lens. When the pressure is released the liquid returns to the reservoir. The whole system may be sealed off from the interior of the eye by a membrane/transparent lens.

As one of average skill in the art will appreciate, the term “substantially” or “approximately”, as may be used herein, provides an industry-accepted tolerance to its corresponding term. Such an industry-accepted tolerance ranges from less than one percent to twenty percent and corresponds to, but is not limited to, component values, integrated circuit process variations, temperature variations, rise and fall times, and/or thermal noise. As one of average skill in the art will further appreciate, the term “operably coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. As one of average skill in the art will also appreciate, inferred coupling (i.e., where one element is coupled to another element by inference) includes direct and indirect coupling between two elements in the same manner as “operably coupled”. As one of average skill in the art will further appreciate, the term “compares favorably”, as may be used herein, indicates that a comparison between two or more elements, items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1.

Although the present invention is described in detail, it should be understood that various changes, substitutions and alterations can be made hereto without departing from the spirit and scope of the invention as described by the appended claims.

What is claimed is:

1. An ocular implant, comprising:
an intraocular lens (IOL) operable to pass optical energy, the IOL comprising:
a first optical membrane;
a second optical membrane;
a liquid located between the first optical membrane and second optical membrane;
at least one liquid reservoir operable to add liquid to or withdraw liquid from the liquid located between the first optical membrane and second optical membrane;
a diaphragm that interfaces ciliary muscles of an eye to the at least one liquid reservoir, wherein the contraction or relaxation of the ciliary muscles forces the at least one reservoir to add liquid to or withdraw liquid from the liquid located between the first optical membrane and second optical membrane; and
a plurality haptics coupled to the IOL operable to position the IOL within an eye.

2. The ocular implant of claim 1, wherein the liquid comprises a high surface tension liquid surrounded by phobic liquid.

3. The ocular implant of claim 1, wherein the contraction or relaxation of the ciliary muscles changes an internal pressure of the liquid located between the first optical membrane and second optical membrane, the internal pressure affecting the curvature of the first optical membrane and the second optical membrane.

4. The ocular implant of claim 1, further comprising a membrane/transparent lens operable to isolate the ocular implant from the interior of the eye.

5. The ocular implant of claim 1, wherein the haptics are angled at about 2.2° to the plane of the IOL.

6. The ocular implant of claim 1, wherein the IOL is operable to replace a natural lens of the eye.

7. The ocular implant of claim 1 operable to be implanted within a sub 2.1 mm incision.

8. The ocular implant of claim 1, wherein the IOL comprises a biconvex optic.

9. An ocular implant, comprising:
an intraocular lens (IOL) operable to pass optical energy, the IOL comprising:
a high surface tension liquid;
a phobic liquid surrounding the high surface tension liquid;
at least one liquid reservoir operable to add or withdraw high surface tension liquid;
a first diaphragm that interfaces ciliary muscles of an eye to the at least one liquid reservoir, wherein the contraction or relaxation of the ciliary muscles force the at least one liquid reservoir to add or withdraw high surface tension liquid; and
a plurality haptics coupled to the IOL operable to position the IOL within an eye.

10. The ocular implant of claim 9, wherein the contraction or relaxation of the ciliary muscles changes an internal pressure of the high surface tension liquid, the internal pressure affecting the curvature of an optical surface of the high surface tension liquid.

11. The ocular implant of claim 9, further comprising a membrane/transparent lens operable to isolate the ocular implant from the interior of the eye.

12. The ocular implant of claim 9, further comprising:
at least one additional liquid reservoir operable to add or withdraw phobic liquid;
a second diaphragm that interfaces ciliary muscles of an eye to the at least one additional liquid reservoir, wherein the contraction or relaxation of the ciliary muscles force the at least one additional liquid reservoir to add or withdraw phobic liquid.

13. The ocular implant of claim 12, wherein the contraction or relaxation of the ciliary muscles changes a differential pressure between the high surface tension liquid and the phobic liquid, the differential pressure affecting the curvature of an interface between the high surface tension liquid and the phobic liquid.

14. The ocular implant of claim 9, wherein the IOL is operable to replace a natural lens of the eye.

15. The ocular implant of claim 9, wherein the IOL comprises a biconvex optic.

16. A method to correct visual impairment of aphakia comprising:
removing a natural lens from an eye;
inserting an intraocular lens (IOL) through an incision of a capsular bag of the eye, the IOL, the IOL comprises:
a first optical membrane;
a second optical membrane;
a liquid located between the first optical membrane and second optical membrane;
at least one liquid reservoir operable to add liquid to or withdraw liquid from the liquid located between the first optical membrane and second optical membrane;
a diaphragm operable to interface ciliary muscles of the eye to the at least one liquid reservoir, wherein the contraction or relaxation of the ciliary muscles force the at least one liquid reservoir to add liquid to or withdraw liquid from the liquid located between the first optical membrane and second optical membrane; and
a plurality haptics coupled to the IOL operable to position the IOL within an eye;
positioning and securing the IOL within the eye with the plurality of haptics coupled to the IOL; and
interfacing the ciliary muscles of the diaphragm of the IOL.

17. The method of claim 16, wherein the liquid comprises a high surface tension liquid.

18. The method of claim 16, wherein the contraction or relaxation of the ciliary muscles changes an internal pressure of the liquid located between the first optical membrane and second optical membrane, the internal pressure affecting the curvature of the first optical membrane and the second optical membrane.

19. The method of claim 16, further comprising isolating the ocular implant from the interior of the eye with a membrane/transparent lens.

20. The method of claim 16, wherein the IOL is operable to replace a natural lens of the eye.

21. The method of claim 16, wherein the IOL comprises a biconvex optic.

22. A method to correct visual impairment of aphakia comprising:
removing a natural lens from an eye;
inserting an intraocular lens (IOL) through an incision of a capsular bag of the eye, the IOL, the IOL comprises:
a high surface tension liquid;
a phobic liquid surrounding the high surface tension liquid;
at least one liquid reservoir operable to add or withdraw high surface tension liquid;
a first diaphragm that interfaces ciliary muscles of an eye to the at least one liquid reservoir, wherein the contraction or relaxation of the ciliary muscles force the at least one liquid reservoir to add or withdraw high surface tension liquid; and
a plurality haptics coupled to the IOL operable to position the IOL within an eye;
positioning and securing the IOL within the eye with the plurality of haptics coupled to the IOL; and
interfacing the ciliary muscles of the diaphragm of the IOL.
